(19) United States
(12) Patent Application Publication
(10) Pub. No.: US 2011/0265202 A1
(43) Pub. Date: Oct. 27, 2011

(54) ENGINEERING OF PLANTS TO EXHIBIT SELF-COMPATIBILITY

(76) Inventors: Vernonica Franklin-Tong, Worcestershire (GB); Frederick Franklin, Worcestershire (GB); Bernardus De Graaf, Cardiff (GB)

(21) Appl. No.: 13/130,965

(22) PCT Filed: Nov. 24, 2009
(86) PCT No.: PCT/GB09/02742

§ 371 Date: Jun. 29, 2011

(30) Foreign Application Priority Data
Nov. 28, 2008 (GB) 0821212.5
Oct. 22, 2009 (GB) 0918528.1

(51) Int. Cl. A01H 1/02; C12N 5/10; A01H 5/10; A01H 5/02; C12N 15/82; A01H 5/00

(52) U.S. Cl. 800/260; 800/278; 800/298; 800/323; 435/419

(57) ABSTRACT

Self-incompatibility (SI) of the common field poppy (Papaver rhoeas) depends on interaction of a pollen transmembrane protein with a pistil ligand protein both encoded by multi-allelic genes at the S locus. Such a locus can be used to confer SI on other plant species.

### FIELD OF THE INVENTION

The majority of flowering plants are hermaphrodites; they possess both male and female reproductive tissues closely adjacent. As a consequence, they generally undergo self-pollination and fertilization. Self-incompatibility (SI) is a genetically-controlled mechanism used by some species of flowering plants to prevent self-pollination or pollination by a genetically-related plant. As a result, these species are naturally out-crossing. The present invention relates to the establishment that a multi-allelic pollen-expressed gene, PrpS, of the common field poppy (Papaver rhoeas) together with a previously identified multi-allelic pistil-expressed gene (the pistil S gene) which, together, are responsible for the SI system of that species. Thus, use of those genes alone, or equivalent genes of related Papaver species, is now shown to confer SI on plants which do not normally possess a SI system.

### BACKGROUND TO THE INVENTION

Three mechanistically distinct SI systems have previously been identified in other genera of plants: Brassicaceae, Solanaceae, Rosaceae and Plantaginaceae (Takayama and Isogai, (2005) Ann. Rev. Plant Biol. 56, 467-489; McClure et al. (2006) Planta 224, 233-245). An SLF—RNase system has also been disclosed in Petunia inflata (Huang US2005/0246788). All these involve an allele-specific interaction between a pistil (female) protein and a corresponding pollen (male) protein both of which are encoded by genes residing at a single genetic locus, referred to as the S locus. This highly specific interaction results in inhibition of pollen tube growth, thus preventing self-pollination. The pistil and pollen S genes exist in different allelic forms which control pollination specificities. Thus, a plant carrying the S₁ allele cannot pollinate itself or another S₁ plant but can pollinate plants of the same species carrying different S alleles.

Although the three known SI systems exhibit similarities in their genetic control, they have evolved independently and, as noted above, are mechanistically distinct. While it was hoped that characterisation of these systems would enable SI to be engineered into plants, so far this objective has not been achieved. The inventors have now elucidated that the SI system of the common poppy depends on pollen membrane receptor-pistil small protein ligand interaction and can be engineered into plants by simply introducing a pair of S gene alleles encoding pistil and pollen proteins which interact.

Pistil S alleles of Papaver rhoeas have previously been cloned and the expression product of such an allele has been shown to interact with incompatible pollen, triggering a Ca²⁺-dependent signalling network, resulting in the inhibition of pollen tube growth and programmed cell death (Foote et al. (1994) Proc. Natl. Acad. Sci. USA 91, 2265-2269; Thomas and Franklin-Tong (2004) Nature 429, 305-309; Thomas et al. (2006) J. Cell Biol. 174, 221-229). Sequence information for the P. rhoeas pistil S₁, S₃ and S₃ alleles is available from databases as detailed below:
Pistil S₁: GenBank accession no. X74333 (Foote et al. (1994) ibid);
Pistil S₃: EMBL, GenBank and DDBJ databases: accession numbers X87100 and X87101 (Walker et al. (1996) Plant Molecular Biology 30, 983-994); and
Pistil S₈: EMBL #AJ005741 (Kurup et al. (1998) Sexual Plant Reproduction 11, 192-198).

Although the inventors previously also identified a pistil S protein-binding glycoprotein in pollen, subsequent studies indicated that it was not the pollen S determinant, but that it might modulate the SI response (Hearn et al. (1996) The Plant Journal 9, 467-475). More recent analysis of a genomic A clone of a limited region surrounding the pistil S₁ allele of Papaver rhoeas also failed to reveal a candidate for the pollen S₁ gene (Wheeler et al. (2003) J. Exp. Bot. 54, 131-139). However, by probing a genomic cosmid library from the S locus of S₁S₃ Papaver rhoeas using pistil S₁ cDNA, the inventors have now identified the corresponding pollen S₁ gene (designated PrpS₁: Papaver rhoeas pollen S₁) in a 42 kb region and cloned a S locus segment that encodes both the Papaver rhoeas pistil and pollen S₁ genes separated at their 3′ ends by only 467 base pairs (see FIG. 5a). Two further Papaver rhoeas pollen S alleles (PrpS₈ and PrpS₃) were subsequently identified from pollen cDNA and genomic DNA of S₃S₈ plants using a PrpS₁ primer (the oligonucleotide of SEQ. ID no. 1 as set out in Example 1), consistent with the polymorphism to be expected of a S locus component for SI.

The mRNA sequences for the PrpS₁ and PrpS₈alleles have been made available in the EMBL/NCBI database with accession numbers AM743176 and AM743177, but it is the studies now reported herein which established the utility of those sequences together with the appropriate previously identified pistil S alleles (S₁ and S₈ respectively) for conferring SI on genetically-engineered plants.

The identified PrpS alleles encode an approximately 20 kDa protein with (at least) three predicted transmembrane domains and with no homology to proteins in existing databases. PrpS protein was shown to be associated with plasma membrane consistent with the proposal that it is a transmembrane protein. As there are no homologues, the Papaver pollen S protein represents a distinct class of transmembrane protein.

Evidence that the PrpS₁, PrpS₃ and PrpS₈ alleles are involved in SI was obtained by confirming tight genetic linkage with the pistil S₁, S₃ and S₈ alleles respectively by segregation analysis using gene-specific primers (see FIG. 7) and by using PrpS allele specific antisense oligonucleotides to alleviate inhibition in vitro of incompatible pollen by recombinant pistil S protein. Moreover, a peptide based on a predicted external domain portion of the PrpS, protein was also shown to be capable of blocking the inhibition of pollen tube growth (inhibition) by recombinant S protein in an in vitro SI assay.

Given the postulated key receptor-ligand interaction responsible for poppy SI, it was hypothesised that just two poppy genes may suffice to transfer SI into other plant species thus overcoming previous problem in achieving this highly desired goal. Significantly, it has been shown using the cruciferous model plant Arabidopsis thaliana (a self-compatible species, not closely-related to poppy), that a PrpS allele and its corresponding pistil S allele when engineered into such plants are indeed sufficient to confer SI. Thus, Arabidopsis pollen expressing PrpS₁ as a fluorescent fusion protein, when grown in vitro, was shown to be inhibited by addition of the corresponding recombinant pistil S₁ protein. Moreover, transgenic Arabidopsis plant lines co-expressing the same pistil and pollen S proteins exhibited reduced male fertility owing to self-pollination inhibition. These experiments provided proof of principle that it is be possible to use a Papaver S locus to engineer SI into other unrelated plants which do not normally possess a SI system.

Further studies confirmed this. In Arabidopsis plant lines transformed with vectors to express the PrpS₁ gene, the percentage germination and mean length of pollen tubes were not significantly different from the wild type. Hence, the inserted genes did not affect the behaviour of the pollen grains, that is, pollen hydration and germination were as normal wild type. The inserted PrpS₁ gene linked to GFP did not interfere with the normal functioning of pollen grains. In fact, no transgenic pollen tubes expressing PrPS₁ germinated in the presence of active S₁ proteins, and this was a highly significant result. In the presence of PrsS₁ proteins, no transgenic tubes that expressed GFP germinated because they expressed the PrPS₁ gene (FIG. 10). This is thought to be due to the PrPS₁ interacting with the PrsS₁ proteins in the growth medium, resulting in the arrest of pollen tube growth.

This data has also now been replicated in vivo, showing that pollen bearing PrPS₁ formed pollen tubes from wild type stigmas, but not from cell lines (designated herein as HZ cell lines) also expressing the S₁ allele (as PrsS₁).

Therefore, the poppy SI system has been introduced into the A. thaliana model and only worked in the presence of its active complementary PrsS protein. This confirms that the poppy PrPS gene is functional in A. thaliana. In other words, the recombinant poppy pistil PrsS₁ protein and the pollen protein, PrpS₁ expressed in A. thaliana were alone sufficient to induce S-specific pollen inhibition in A. thaliana.

In turn, this suggests that the events downstream of the interaction are well conserved in unrelated species and are, therefore, the present SI system will likely function in a broad range of plant species.

### SUMMARY OF THE INVENTION

In one aspect, the present invention thus provides a method of obtaining plants which exhibit self-incompatibilty (SI), or in which SI is inducible, which comprises transforming plants or cultured plant cells with both (i) a pollen S allele of a Papaver S locus or a functional variant thereof and (ii) a pistil S allele of said Papaver S locus or a functional variant thereof, said pollen and pistil S alleles encoding respectively pollen and pistil proteins of a Papaver SI system which prevents self-pollination, and, if need be, further generating plants from transformed cultured cells.

Such transformation may be followed by controlled pollination of transformed plants to obtain plants stably transformed and homozygous for the pollen S allele or functional variant thereof and the pistil S allele or functional variant thereof.

At its simplest, the SI system is a 2-component system comprising the pollen S allele (or a variant) and the pistil S allele (or a variant).

Engineered plants thus obtained which exhibit SI, or in which SI is inducible, constitute further aspects of the invention. Thus, the present invention provides a plant engineered to express both a pollen S allele and the corresponding pistil S allele of a Papaver S locus, or an equivalent plant wherein one or both of said alleles is substituted by a functional variant, whereby SI is maintained or is inducible.

Such a functional variant may comprise a cDNA sequence, e.g. a cDNA which retains coding sequences for both the pistil S protein and pollen S protein of a native Papaver S locus. Conveniently, however, a complete native Papaver S locus, e.g. an S locus of P. rhoeas or P. nudicale, may be employed. For example and preferably, the S₁ locus providing both the PrpS₁ and pistil S₁ alleles of P. rhoeas may be employed, which as noted above can be advantageously isolated in a S locus genomic DNA with less than 500 by 3′ end separation. However, it will be appreciated that native pistil and pollen S alleles may alternatively be isolated as separate alleles and provided as a cassette comprising a synthetic S locus. This may be preferred where the native pistil and pollen S alleles are separated by far greater then 500 by such as found for the P. rhoeas PrpS₃ and pistil S₃ allele pair and the PrpS₈ and pistil S₈ allele pair (see Example 1). As indicated above, initial introduction into a plant of an S locus may be followed by controlled pollination to obtain a plant line stably transformed and homozygous for that locus.

As also indicated above, the chosen alleles to provide SI may have native promoters substituted by inducible promoters, e.g. such that SI can be turned on by chemical spraying.

Such transfer of SI into plants that do not normally possess SI has a number of potentially important uses, for example:
i. Many crop species do not possess SI systems, or in some cases have been selected so that they are no longer functional. This represents a significant problem for plant-breeders and seed companies who make widespread use of and sell F₁-hybrid varieties, which generally have better characteristics than their parents. This is because the production of F1 seed in the absence of SI is dependent on laborious and time-consuming hand emasculation of individual plants to prevent self-pollination. Introduction of a Papaver SI system into such plants would obviate the use of hand-emasculation and make production of F1 hybrids easier and cheaper. If a crop species is self-incompatible, then it can be crossed without any emasculation, as no pollen can fertilise the originating plant. Obtaining of F1 hybrid seed from plants engineered to express an SI system in accordance with the invention, and use of such seed to obtain F1 hybrid plant varieties, thus represents a potentially commercially important further aspect of the invention.
ii. Once a flowering plant has been pollinated, a senescence pathway is induced and the petals are rapidly shed. This has a significant effect on the “shelf-life” of ornamental cut flowers or plants and is of considerable horticultural economic importance. However, if self-pollination is blocked by SI, the senescence pathway will not be activated, thus prolonging shelf-life of ornamental plants or cut flowers

As noted above, the Papaver rhoeas pollen S gene (PrpS) displays the polymorphism typical of an SI component. Such a coding sequence may be transcriptionally-linked to its native pollen promoter for directing expression in pollen, e.g. as part of a complete native S locus for use in engineering SI into plants as discussed above, or a heterologous promoter, which may be tissue specific. Such a heterologous promoter may be inducible. It will be appreciated that cells engineered using such a DNA to express a pollen S protein or functional analogue thereof may be subjected to targeted cell ablation by contact with, or co-expression of, a pistil S protein or functional analogue thereof whereby SI system-type induced programmed cell death (PCD) is induced. Such targeted cell ablation constitutes a further aspect of the invention and may be employed in ectopic tissues and, for example, in removing pollen from a mixture of pollens.

The alleles are preferably present in a complete native Papaver S locus. A PrpS and pistil S allele pair of Papaver rhoeas are preferably employed. More preferably, the PrpS₁ and pistil S₁ allele pair of P. rhoeas are employed. The plants or cells to be transformed may contain, or may be simultaneously transformed with, a transgene for a further desired characteristic.

The method may also further comprise controlled pollination of transformed plants to obtain plants stably transformed and homozygous for said pollen S allele or a functional variant thereof and said pistil S allele or a functional variant thereof. Preferably, said further comprises crossing said plants homozygous for said pollen S allele or a functional variant thereof and said pistil S allele or a functional variant thereof to obtain F1 hybrid seed and optionally growing from said seed F1 hybrid plants. F1 hybrid seed thereby obtained and F1 hybrid plants derived therefrom are provided.

Also provided is a kit of polynucleotides, for instance for use in the present method. Said kit may comprise two polynucleotides. The first polynucleotide may have a pollen S allele of a Papaver S locus or a functional variant thereof. The second polynucleotide may have a pistil S allele of said Papaver S locus or a functional variant thereof. The pollen and pistil S alleles encode, respectively, pollen and pistil proteins of a Papaver SI system which prevents self-pollination. The polynucleotides may be separate, i.e. different chains, or may be part of the same polynucleotide chain, compassing both the pistil and pollen alleles or variants thereof. Suitable promoters for expression of the corresponding S alleles in the pistil or pollen are preferably provided.

The kit may also comprise a vector comprising said both polynucleotides, or two vectors each comprising one or other of said polynucleotides. The kit may also comprise instructions for use.

Transformation may be achieved using standard plant vectors, such as Agrobacterium, or delivery by a gene gun, for instance where the polynucleotide(s) are attached to gold particles. Where Agrobacterium is used, transformation of plants by floral dip is preferred.

Plants obtained by the present method and seeds produced therefrom, are also provided. Indeed, plants grown from these seeds are also provided, as are any plants grown or transformed to comprise the present SI system provided said plants are not Papaver in which the system occurs naturally.

A plant obtained by the present method, which exhibits SI, or in which SI can be induced, by virtue of said pollen S allele or functional variant thereof and said pistil S allele or functional variant thereof, is also provided. The plant may be homozygous for said alleles conferring SI. Cultured plant cells transformed in accordance with the invention are provided as well.

The plant may be an ornamental plant. Seed therefor and cut flowers derived therefrom are provided. The plants in which the present SI system may be used can be monocots or dicots. Preferred are ornamental flowers and crops. Crop plants of interest may include, but are not limited to, soybean (including the variety known as Glycine max), cotton, canola (also known as rape), corn (also known as maize and Zea mays), wheat, sunflower, sorghum, alfalfa, barley, millet, rice, fruit and vegetable crops.

The invention will be further described below with reference to the following listed figures.

### BRIEF DESCRIPTION OF THE FIGURES

FIG. 1: (a) the P. rhoeas PrpS₁ full length cDNA in which the translation start codon (ATG) and translation stop codon (TAA) are shown in bold underlined (SEQ. ID no. 2) and (b) the corresponding PrpS, protein sequence (SEQ. ID no. 3).

FIG. 2: (a) the P. rhoeas PrpS₃ coding cDNA sequence in which the translation start codon (ATG) and translation stop codon (TAA) are shown in bold underlined (SEQ. ID no. 4) and (b) the corresponding PrpS₃ protein sequence (SEQ. ID no. 5).

FIG. 3: (a) the P. rhoeas PrpS₈ full length cDNA sequence in which the translation start codon (ATG) and translation stop codon (TAA) are shown in bold underlined (SEQ. ID no. 6) and (b) the corresponding PrpS₈ protein sequence (SEQ. ID no. 7).

FIG. 4: P. rhoeas pistil S allele cDNAs (S₁, S₃, and S₈) and the corresponding deduced amino acid sequences (SEQ. ID nos. 8 to 13)

FIG. 5:

(a) Organization of the S₁ locus of P. rhoeas. Blocked arrows indicate the S₁ and PrpS₁ coding sequences and their orientation; transcription start site (+1).

(b) Alignment of PrpS₁, PrP₃ and PrpS₈ deduced amino acid sequences (SEQ. ID no. 3, SEQ ID no. 5 and SEQ. ID no. 7 respectively);
* indicates identical amino acids;
. indicates hypervariable amino acids across all 3 alleles; and
: indicates 2 out of 3 amino acids are conserved.

FIG. 6: RT-PCR Segregation analysis described in Example 2 showing the linkage of PrpS to the cognate pistil S in crosses of the segregating families 1,3/3,8 and 1,8/3,8.

FIG. 7:

(a) Effect of PrpS₁ extracellular domain peptide added to SI-induced pollen from plants carrying S₁S₃ alleles. The peptide “rescued” pollen from inhibition (middle panel); randomized peptide 2 had no effect (right panel). Left panel shows typical SI induced inhibition of S₁S₃ pollen in the presence of S₁ and S₃ proteins but in the absence of PrpS₁ peptide.

(b) Effect of PrpS₁ and PrpS₈ antisense oligonucleotides (as-ODNs: as-PrpS₁, as-PrpS₈) on pollen subject to SI-induced inhibition. The as-ODNs “rescued” pollen, carrying S₁S₃ or S₃S₈ alleles from SI-induced inhibition in an S-specific manner, while PrpS₁ and PrpS₈ sense oligonucleotides (s-ODNs: s-PrpS₁, s-PrpS₈) did not. Controls: untreated pollen and as-ODNs without SI induction controls (white bars); SI-induced pollen (black bars); SI-induced in presence of as-ODN (crosshatched bars); SI-induced in presence of s-ODNs (dotted bars).

FIG. 8: P. rhoeas, expressing the S₁ and S₈ haplotypes, was used to verify whether the S proteins were functional. In the presence of S₁ proteins alone, partial inhibition (p=0.000, n=100×3) was achieved while a combination of S₁ and S₈ proteins completely prevented pollen tubes from growing (p=0.000, n=100×3);

FIG. 9: S proteins had no effect on Col0 (p=0.760, n=100×3). There was no significant difference in the frequency of germination of pollen tubes between Col0 and BG16.8.3 (p=0.400, n=100×3);

FIG. 10: shows the % germination of pollen tubes with different S proteins. There was no significant difference observed in the percentage germination of BG16.8.3 pollen tubes subjected to various treatments (p=0.315, n=100×3);

FIG. 11: the inserted PrPS₁ gene triggered SI in an S-specific manner and prevented the growth of pollen tubes, which expressed GFP only, in the presence of active S₁ proteins. Denatured S₁ protein was used as a control to show that active S₁ protein was responsible for triggering the SI response (p=0.005, n=100×3). S₈ proteins were added as another control to verify that SI was induced in an S-specificity manner (p=0.000, n=100×3);

FIG. 12: the viability of Arabidopsis wild type pollen (blue bars) and pollen expressing PrpS-GFP (yellow bars) at t=0 h and t=2 h in the absence and presence of poppy recombinant female S-determinant PrsS₁;

FIG. 13: the viability of Arabidopsis wild type pollen (black bars) and pollen expressing PrpS-GFP (yellow bars) at t=0 h and t=4 h in the absence and presence of poppy recombinant female determinant PrsS₁; and

FIG. 14: the viability of Arabidopsis wild type pollen (dark green bars) and pollen expressing PrpS-GFP (yellow bars) at t=0 h and t>8 h in the absence and presence of poppy recombinant female determinant PrsS₁.

### DETAILED DESCRIPTION

The genes required to provide the required S locus proteins in an engineered plant of the invention may be introduced into plant cells using conventional techniques such as Ti plasmids, electroporation and gene gun delivery of DNA-coated microparticles, followed by generation of transformed plants, e.g. from transformed cultured cells, again using routine techniques. By way of example, Example 4 illustrates use of Ti plasmids carrying a P. rhoeas S₁ locus and antibiotic resistance gene to transform Arabidopsis plants as referred to above with initial screening of Tₒ seed for presence of the antibiotic resistance gene and generation of transformed plants (T₁) from selected seed carrying the S₁ locus. Controlled pollination may then be carried out to obtain the desired stably transformed plant line, homozygous for the S locus and exhibiting SI. The genes of the S locus may be introduced into plant cells already transformed with a transgene to provide a further desired characteristic or simultaneously with such a transgene.

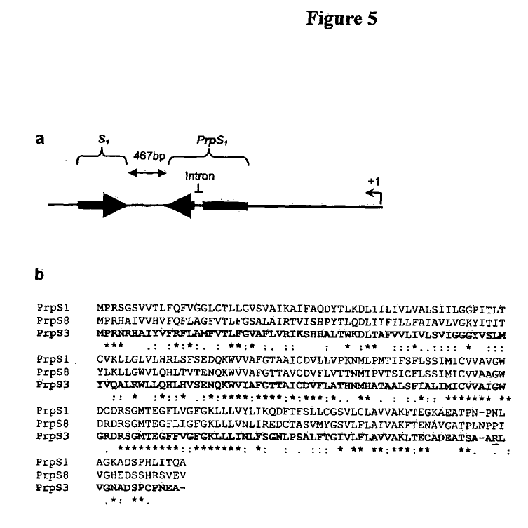

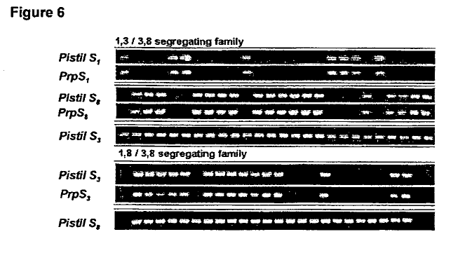

The S locus genes might be transfected separately but generally it will be preferred to introduce a complete S locus, either a S gene expression cassette or a native locus. As indicated above, the S locus may be conveniently a complete native Papaver S locus in a genomic DNA fragment, e.g. a complete native Papaver rhoeas S locus, preferably an S₁ locus providing both the PrpS₁ and pistil S₁ alleles of Papaver rhoeas as shown in FIG. 5a. As detailed in Example 4, such an S₁ locus may be conveniently obtained in a genomic DNA restriction fragment, e.g. a NotI fragment of 6.8 kb, suitable for insertion into a vector such as a Ti plasmid for direct transfection of plant cells.

Whilst any PrpS allele or encoded peptide is preferred, the PrpS₃ or PrpS₈ alleles or encoded peptides are more preferable and the PrpS₁ allele or encoded peptide is particularly preferred. It is therefore expected that any cognate pair would work, for instance PrsS2 and PrpS2 should work as a cognate incompatible pair. Other matching sS and pS alleles should also function in an SI system.

It will be appreciated, however, that either or both of the pollen S allele and the pistil S allele may be replaced by a functional variant whereby SI is maintained or is inducible. The 3 pollen S alleles PrpS₁, PrpS₃ and PrpS₈ have been found to contain an intron, for example the PrpS₁ allele intron of 94 by is found near the 3′ end of the gene (shown in FIG. 5a). It will be recognised therefore that, for example, genomic DNA of a native P. rhoeas S₁ locus may be substituted by a cDNA encoding the same S locus proteins for transformation of plant cells in accordance with the invention. It will also be appreciated that a native promoter may be substituted by a heterologous promoter of the same tissue specificity, e.g. a Papaver pollen S protein coding sequence such as the PrpS₁ allele coding sequence may be transcriptionally linked to the promoter of ntp303, a Nicotiana tabacum gene which is known to be specifically active in pollen and provide higher levels of expression (see Example 4).

In principle, any good pollen or pistil-specific promoter that gives high expression in these tissues in the relevant crop is useful linteh present invention. Suitable promoters may include the Lat52 promoter or the cognate Prps1 promoter, whilst the ntp303 promoter mention above is particularly preferred.

Expression of pistil S may be driven by the STIG1 promoter from Nicotiana tabacum. Homologous promoter sequences from other flowering plant species, including A thaliana, may be employed. One or both of the S locus coding sequences may be varied provided an S locus is retained which provides a pistil protein and pollen protein which interact to provide SI.

Cultured plant cells transformed as above to acquire genes conferring SI on a plant generated therefrom constitute a further aspect of the invention.

The identification of the key protein interaction for SI in P. rhoeas opens the way not only for a variety of Papaver S locus gene pairs to be used to confer SI in engineered plants, but also as already briefly discussed above, use of Papaver pollen S gene coding sequences alone, or co-expressed with an appropriate pistil S gene coding sequence, to enable targeted programmed cell death (PCD) in a plant tissue, using an appropriate tissue-specific promoter.

Thus in a still further aspect of the invention, there is provided a method of targeted plant cell ablation in plant tissue wherein the cells of the tissue express as a heterologous protein the pollen S component of a Papaver SI system or a functional analogue thereof, which comprises contacting said cells with, or co-expressing in said cells, a Papaver pistil S protein or functional analogue thereof whereby programmed cell death occurs. Expression of either or both the pollen S protein and pistil S protein may be under the control of a tissue specific inducible promoter such that S locus-induced PCD may be turned on as desired. A suitable tissue specific inducible promoter is dexamethosone.

Hence, of interest are DNAs wherein the coding sequence for the pollen S component of a Papaver SI system, e.g. a P. rhoeas SI system, is transcriptionally-linked to a heterologous promoter suitable for directing expression in a specific plant tissue, which may be pollen or a non-pollen tissue. Such DNAs may also encode a pistil S protein or functional analogue thereof whereby the DNA provides a complete S locus which causes, or can be induced to cause, S locus-induced programmed cell death. As indicated above, by means of choice of promoter it is envisaged that such S locus-induced PCD may be provided in inducible manner to target ectopic tissues.

Such a method may also be used to target pollen carrying a transgene for expression of the pollen S component of a Papaver SI system and may find use in identifying further such genes and as previously noted above inhibiting pollen in a mixture of pollens.

Example 14, for instance, is useful in determining when or if key features of SI have been triggered in the target plant. Example 16 shows how to determine if the present SI system is functional in vivo in barley, an important example of a crop plant.

The following examples illustrate the invention.

### EXAMPLES

### Example 1

### Cloning & Characterisation of PrpS₁, PrpS₃ and PrpS₈

Summary

A genomic clone of PrpS₁ was identified by nucleotide sequence analysis of a cloned 42 kb fragment carrying the S₁ locus, obtained by screening a Papaver rhoeas S₁S₃ cosmid library with the pistil S₁ cDNA. A PrpS₁, PrpS₃ and PrpS8 cDNA clone were subsequently obtained using a combination of RT-PCR, 5′-RACE and 3′-RACE PCR.

Detailed Methods

Cloning and Sequence Analysis

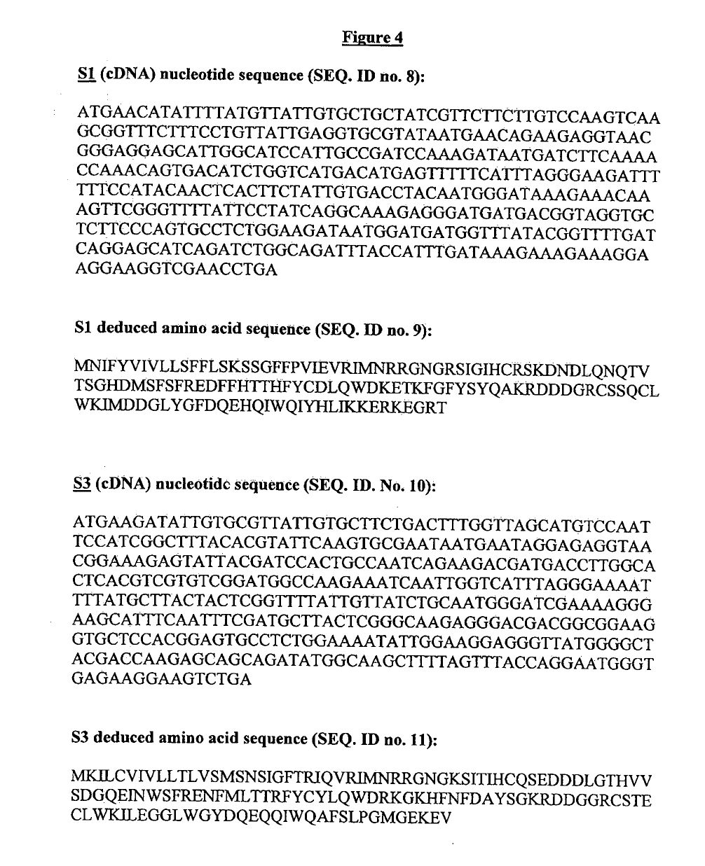

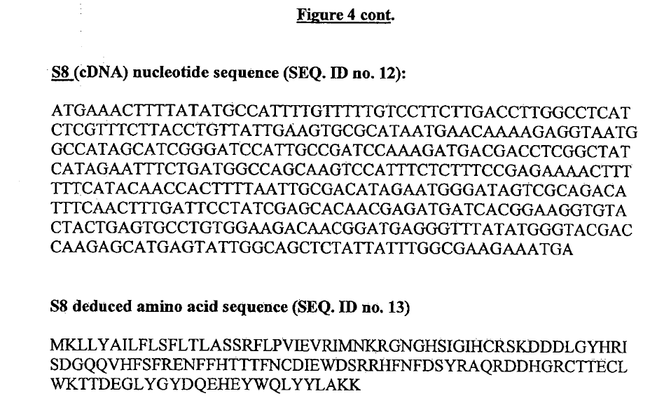

A genomic DNA library from Papaver rhoeas S₁S₃ plants was constructed in SuperCos1 (Stratagene) following the manufacturer's instructions. The library was constructed by BamHI/XbaI digestion and cloning into the BamHI/XbaI sites of SuperCos1. Screening the library with pistil S₁ cDNA (see FIG. 4; SEQ. ID no. 8) resulted in the isolation of a 42 kb fragment containing the pistil S₁ allele. The DNA upstream and downstream of the pistil S₁ allele was sequenced and analyzed using BLAST (http://ncbi.nlm.nih.qov/BLAST), ORF Finder (http://searchlauncher.bcm.tmc.edu) [Worley et al. (1995) Genome Res. 5 173-184: An enhanced BLAST-based search tool that integrates multiple biological information resources into sequence similarity search results] and TMHMM (http://www.cbs.dtu.dk/services/TMHMM) [Krogh et al. (2001) J. Mol. Biol. 305, 567-580: Predicting transmembrane protein topology with a hidden Markov model: application to complete genomes]. The presence and organization of the final S₁ locus sequence was confirmed by PCR on genomic DNA of S₁ and non-S₁ containing plants.

The PrpS₈ allele was obtained from pollen cDNA and genomic DNA of S₃S₈ plants by standard PCR techniques including 5′ and 3′ RACE using a PrpS₁ primer (GCGACCGAAGTGGCATG; SEQ. ID. No.1) at low annealing temperatures (48° C.). It was expected that further alleles of PrpS could be quite divergent from PrpS₁ so 8 different primers were designed against regions of the sequence. 3′ RACE was carried out using all 8 primers and then PCR products blotted and probed with PrpS₁. Products amplified using one primer hybridised most strongly to the PrpS₁ probe and therefore this primer was selected for use in further experiments.

To obtain PrpS₃, degenerate primers were designed using PrpS₁ and PrpS₈ sequences (9 primer set combinations). This was followed by use of 3′ and 5′ Race primers to obtain and confirm full length PrpS₃

Expression Analysis

Standard PCR techniques including 3′ and 5′ RACE were used for expression analysis. Anthers from plants carrying the S₁ allele at different stages of flower development were collected and total RNA was extracted using the RNAeasy plant mini kit (QIAGEN) following the manufacturer's instructions. After DNase treatment cDNA was synthesized using the standard Omniscript RT protocol (QIAGEN). PCR with primers against Papaver rhoeas glyceraldehyde-3-phosphate dehydrogenase (GAPD) transcripts was used as a control; gene-specific primers were used to amplify PrpS₁ transcripts using 30 PCR cycles.

Production of Antisera

The predicted 60 amino acid cytoplasmic C-terminus of PrpS₁ was expressed as a HIS-tagged recombinant protein using pET21b (Novagen). Recombinant protein was isolated from E. coli (BL21) using Ni-NTA resin following the manufacturer's (QIAGEN) protocol. Antisera (PrpS1-60C) was raised in rats (ISL Immune Systems, Paignton, UK).

Protein Extraction for SDS-PAGE and Western Blotting

Extracts enriched for membrane proteins from PrpS₁-containing pollen grown in vitro for 30 mins, leaf, stigma and root tissue were made with 100 mM Tris-HCl, 200 mM NaCl, 2 mM EDTA, 1 M sucrose, 0.5% Triton X-100, and a cocktail of protease inhibitors (Roche Ltd) and protein concentrations were determined. Proteins were separated by 12.5% SDS-PAGE with a Mini-PROTEAN III system (BioRad) using standard procedures (Laemmli (1970) Nature 227, 680-685) and electroblotted (3 hrs at 400 mA). Hybond C membranes (GE Healthcare) were incubated with the PrpS1-60C antibody (1:2000) for 2 hrs and alkaline phosphatase conjugated anti-rat secondary antibody (Sigma), and NBT/BCIP substrate used for detection.

Immuno-Localisation

Papaver rhoeas pollen (S₁/S₃) was grown in germination medium (GM; Snowman et al. (2002) Plant Cell 14, 2613-2626) for 1 hr at 25° C. before fixation with 2% paraformaldehyde for 1 hr. Pollen tubes were collected and washed three times in Tris-buffered saline pH 7.4 (TBS), then in 15 mM MES buffer pH 5.0 before being treated for 10 mins with 0.05% cellulase and 0.05% macerozyme in MES, 0.1 mM PMSF and 1% BSA. 0.1% Triton X-100 in TBS was added for 10 mins and pollen tubes incubated with blocking buffer (TBS+1% BSA) for 30 min, followed by incubation with the PrpS1-60C antibody (1:500 in TBS+1% BSA) at 4° C. overnight and then FITC-conjugated goat anti-rat antibody (1:50) for 1.5 hrs. Pollen tubes were mounted on glass slides with Vectashield (Vector Laboratories, USA) and imaged using confocal microscopy (Bio-Rad Radiance 200MP; Nikon Diaphot). Single scan images were collected with a 100× plan-Apo 1.4 NA oil objective (Nikon). When preimmune antiserum was used instead of primary antibody, no signal was obtained using identical additions and settings.

Discussion

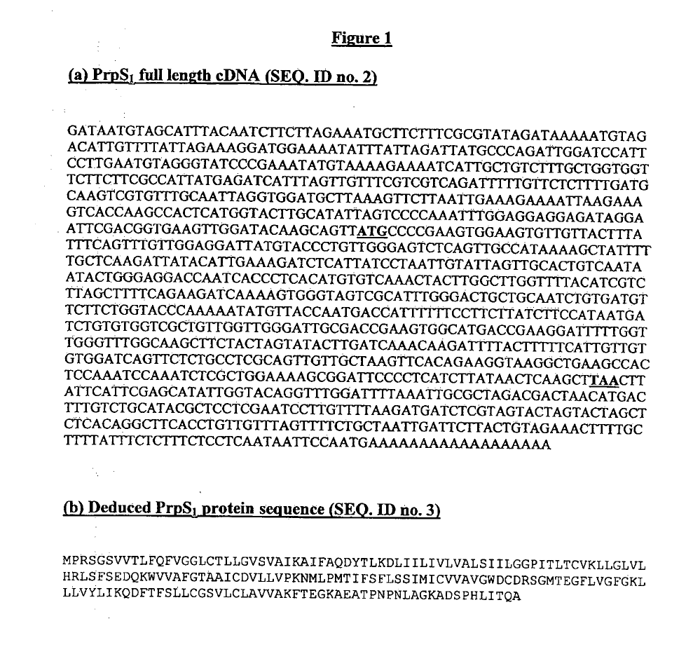

Analysis of the cosmid clone comprising the 42 kb region of the S₁ locus first identified a novel putative open reading frame (ORF) in the vicinity of the S₁ pistil gene (see FIG. 5a). Expression analysis using RT-PCR revealed that the ORF was specifically transcribed in pollen, with maximum expression around anthesis. The temporal expression pattern is very similar to that of the pistil S gene (Foote et al. (1994) ibid). These data suggested that the ORF was a candidate for a Papaver pollen S gene (designated PrpS₁). The genomic copy of PrpS₁ comprises 1206 by and has a predicted 94 by intron near the 3′ end of the gene (see FIG. 5a). The full length corresponding cDNA (FIG. 1a) obtained using RT-PCR, 3′ and 5′ RACE revealed a 579 by ORF encoding a protein with a predicted Mᵣ of 20.5 kDa, pI 7.55 (FIG. 1b).

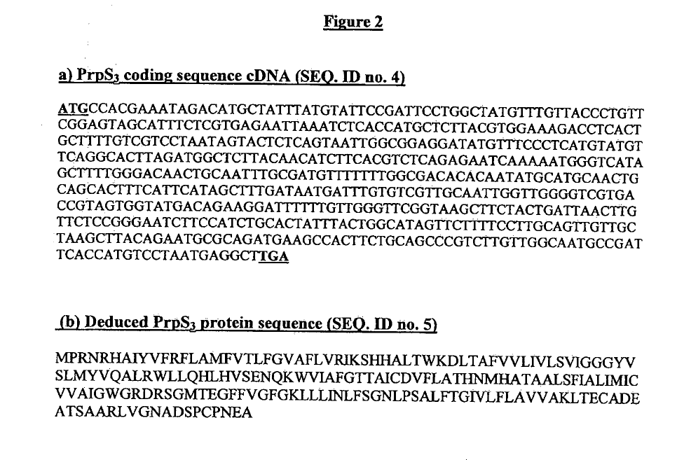

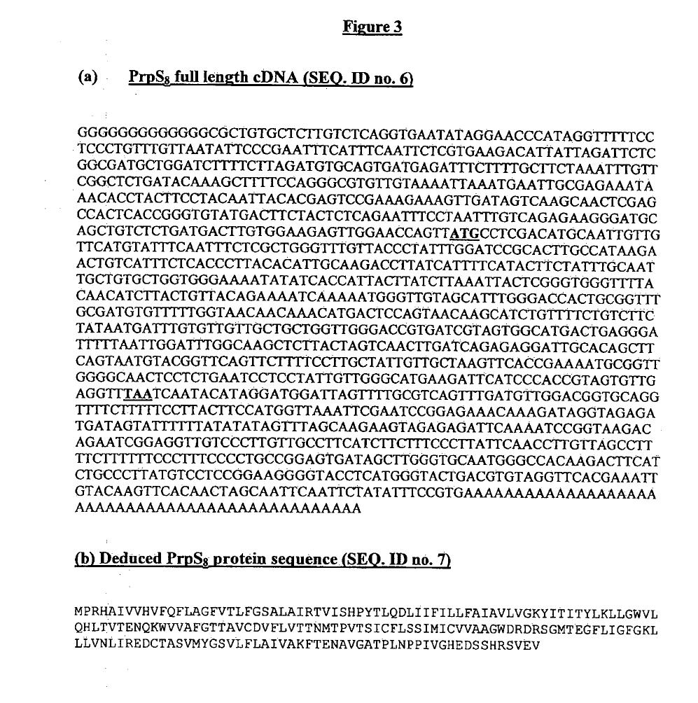

The subsequently cloned PrpS₈ cDNA coding region was found to be 582 by as shown in FIG. 3a and encode a protein of predicted Mᵣ 21.1 kDa, pI 6.57 (FIG. 3b). As noted above, a high level of sequence polymorphism is a well-documented feature of other S locus proteins; S alleles have unusually high amino acid sequence divergence within species. Papaver rhoeas is no exception with the pistil S₁ and S₈ proteins having 63.7% identity (Kurup et al. (1998) Sexual Plant Reproduction 11, 192-198). PrpS also exhibits the level of polymorphism typical of an S locus product since the full length PrpS₁ and PrpS₈ sequences are only 58.9% identical at the amino acid level (see FIG. 5b). As indicated above, details of the cloned PrpS₃ cDNA coding region are shown in both FIG. 2a and FIG. 5b.

A key requirement to maintain a functional SI system is that the pistil and pollen components must be genetically tightly linked at the S locus. As noted above, sequence analysis of the S₁ cosmid showed that the PrpS₁ and pistil S₁ genes likely fulfill this requirement as their 3′ ends are separated by only 467 by (see FIG. 5a). Analysis of a 6 kb fragment flanking the pistil S₈ gene, obtained by inverse-PCR, did not reveal the presence of PrpS₈. Thus, the physical separation of the S₈ locus genes is greater than that of their S₁ locus counterparts. Considerable variation in the physical distance between S locus genes, up to several hundred kb, has previously been reported in Brassica (Boyes and Nasrallah. (1993) Mol. Gen. Genet. 236, 369-373; Casselman et al. (2000) Plant Cell 12, 23-24). To obtain evidence of genetic linkage between S₈ and PrpS₈, and between S₃ and PrpS₃, segregation analysis was conducted as detailed in Example 2.

PrpS₁ encodes a protein with a predicted Mᵣ of 20.5 kDa, pI 7.55. PrpS₃ and PrpS₈ encode proteins of predicted Mr of 20.9 kDa (pI 8.8) and 21.1kDa (pI 6.57) respectively Sequence analysis indicated that PrpS₁, PrpS₃ and PrpS₈ both have a predicted 35 amino acid extracellular domain and three predicted transmembrane domains (FIG. 5b); the C-terminus is predicted to be cytosolic.

As noted above, neither PrpS₁, PrpS₃ nor PrpS₈ exhibits sequence homology to any protein in existing databases. These data suggest that PrpS is a distinct type of transmembrane protein. In support of this, Western analysis using antisera raised against PrpS₁ revealed that PrpS1 was detected specifically in S₁ pollen membrane-enriched extracts and was not detected in cytosolic extracts. Analysis of Western blots revealed that the antisera detect the predicted ˜20 kDa PrpS product. Immuno-localization studies, investigating the distribution of PrpS₁, revealed a clear association with the pollen plasma membrane. This localization is consistent with the hypothesis that PrpS is a transmembrane receptor for the pistil S protein.

### Example 2

### Linkage Analysis

In summary, linkage analysis was carried out on DNA extracted from individual plants from full-sib families segregating either for the haplotypes S₁S₃ and S₃S₈ or S₁S₈ and S₃S₈ using gene specific primers for both pistil S and PrpS, in order to demonstrate linkage of PrpS with pistil S and, therefore, the S locus.

Method

The genomic DNA from leaf of >30 plants from a single family segregating for S₁S₃or S₃S₈ was extracted using Nucleon Phytopure plant DNA extraction kit (Amersham Biosciences). PCR was carried out on the DNA samples, testing for the presence of the S₁, S₃, S₈ genes and the PrpS₁, Prp₃ and PrpS₈ genes, using gene-specific primers as follows

S₁:
(SEQ. ID no. 14)
5′ primer-GGCATATGTTCTTTCCTGTTATTGAGGTGCGT
(SEQ. ID no. 15)
3′ primer-CCGGATCCTCAGGTTCGACCTTCCTTCC
S₃:
(SEQ. ID no. 16)
5′ primer-CGCATATGATCGGCTTTACACGTATTCAAGTG
(SEQ. ID no. 17)
3′ primer-CCGGATCCTCAGACTTCCTTCTCACCCATTCC
S₈:
(SEQ. ID no. 18)
5′ primer-CTTCTTGACCTTGGCCTCATCTCG
(SEQ. ID no. 19)
3′ primer-CTTCGCCAAATAATAGAGCTGCC
PrpS₁:
(SEQ. ID no. 20)
5′ primer-CAGGATCCGTTGCCATAAAAGCTATTTTTGCTC
(SEQ ID. No. 21)
3′ primer-GAATCCGCTTTTCCAGCGAG
PrpS₃:
(SEQ. ID no. 22)
5′ primer-CATGTGAAGGGAGACACTTGTCAGC
(SEQ. ID no. 23)
3′ primer-CCTAGACACCTAAAATTGTAATGGCTGC
PrpS₈:
(SEQ. ID. No. 24)
5′ primer-GGGCACAGCTTCAGTAATGTACG
(SEQ ID. No. 25)
3′ primer-GGCTGACGCAAAACTAATCCATCC

Results

PrpS₁ was amplified only from plants carrying S₁, PrpS₃ was amplified only from plants carrying S₃ and PrpS₈ was amplified only from plants carrying the S₈ allele. The pistil S alleles S₁, S₃ and S₈ were also amplified only from plants carrying the S₁, S₃ and S₈ alleles respectively; the S₃ allele was amplified from all S₁S₃ and S₃5₈ plants as expected. This demonstrates linkage of PrpS₁, PrpS₃ and PrpS₈, and the pistil genes S₁, S₃ and S₈ to their respective S loci, as there is no recombination (P<0.05%).

### Example 3

### Pollen Inhibition in In Vitro SI Assays

Summary

A 15 amino acid peptide corresponding to part of the predicted external domain of PrpS₁ was tested for ability to block pollen tube growth inhibition in an in vitro SI assay in which pollen was grown on solid germination medium before SI induction using recombinant pistil S protein (Thomas and Franklin-Tong (2004) ibid). To confirm a functional role and allele specificity for PrpS₁ and PrpS₈ in pollen tube inhibition, a gene specific antisense approach was also used with an in vitro SI assay.

Methods

Peptide Bioassay

Based on the TMHMM (http://www.cbs.dtu.dk/services/TMHMM) prediction of PrpS₁, a 15 amino acid residue peptide (DQKWWAFGTAAICD; SEQ. ID no. 26) corresponding to part of the predicted 35 amino acid residue external domain (see FIG. 5b) and two randomized versions of this peptide (1:GVVCAWIFDTAAQKD (SEQ. ID no. 27) and 2: FTVDVKDCAAAWGQI (SEQ. ID no. 28)) were synthesized by Alta Bioscience (University of Birmingham, UK). Using pollen from S₁S₃ P. rhoeas and 5₃S₈ P. rhoeas, pollen inhibition was compared in the presence of S proteins alone (SI-induced), with SI in the presence of PrpS peptide (SI+peptide) or with SI in the presence of randomizd PrpS peptide (SI+control peptide). Each peptide was mixed with recombinant S proteins for 20 mins at room temperature prior to adding to pollen, which was grown for 1 hr in vitro. Pollen grains and tubes were scored according to two categories: “inhibition” or “growth”; a minimum of 100 pollen grains/tubes was scored for each sample. Data were analysed using Fisher's Exact Test for 2×2 contingency tables.

Antisense Oligo Silencing of PrpS Expression.

Phosphorothioated gene-specific antisense oligodeoxynucleotides (as-ODN) and their sense controls (s-ODN) were designed for PrpS₁ and PrpS₈ mRNA:

| | |
|---|---|
| PrpS₁ as-ODN: | |
| gtccTCCCAGTATTAttga | (SEQ. ID no. 29) |
| PrpS₁ s-ODN: | |
| tcaaTAATACTGGGAggac | (SEQ. ID no. 30) |
| PrpS₈ as-ODN: | |
| ttccCACCAGCACAGCaatt | (SEQ. ID. No. 31) |
| PrpS₈ s-ODN: | |
| aattGCTGTGCTGGTGggaa. | (SEQ. ID. No. 32) |

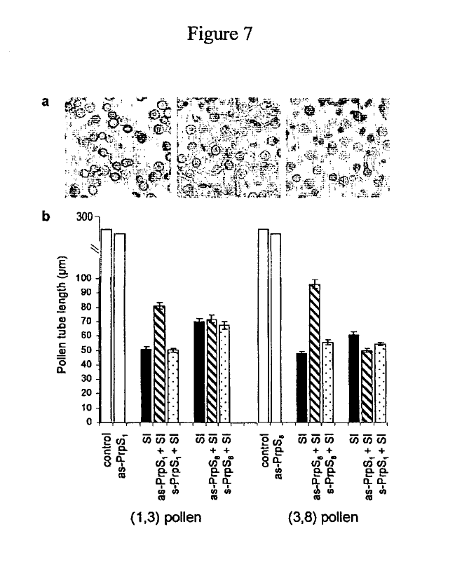

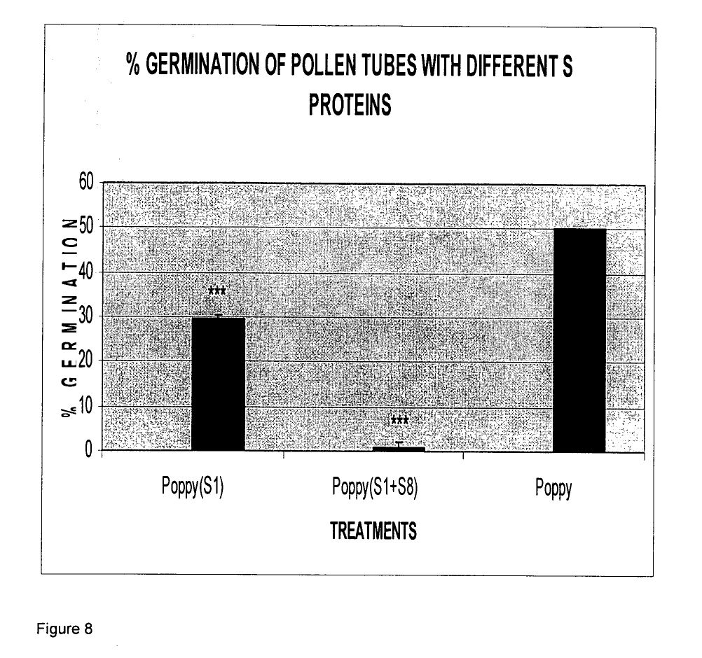

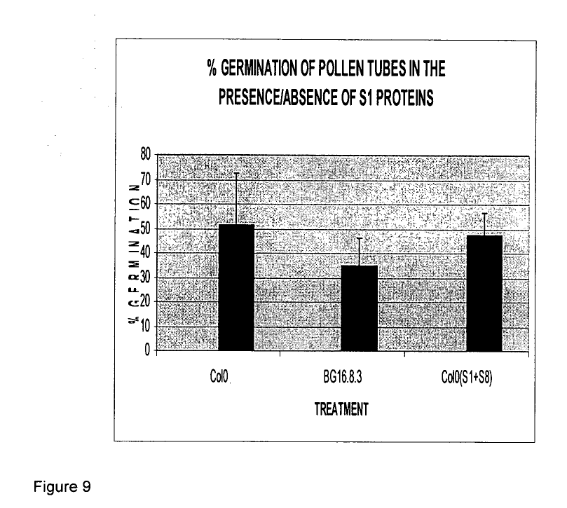

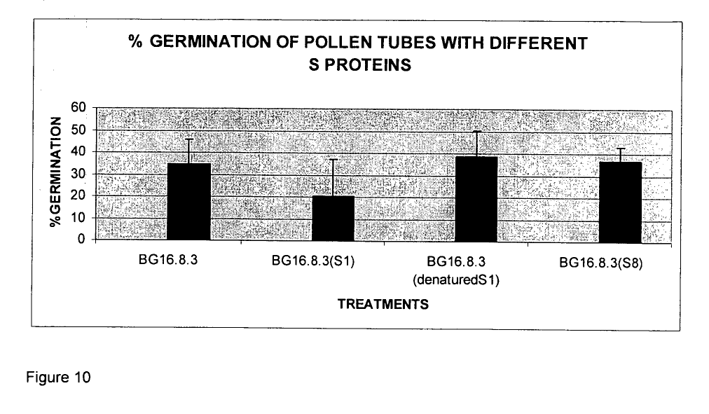

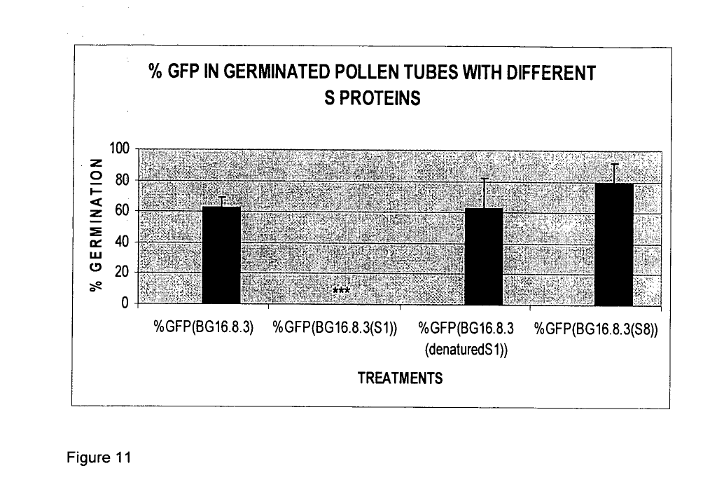

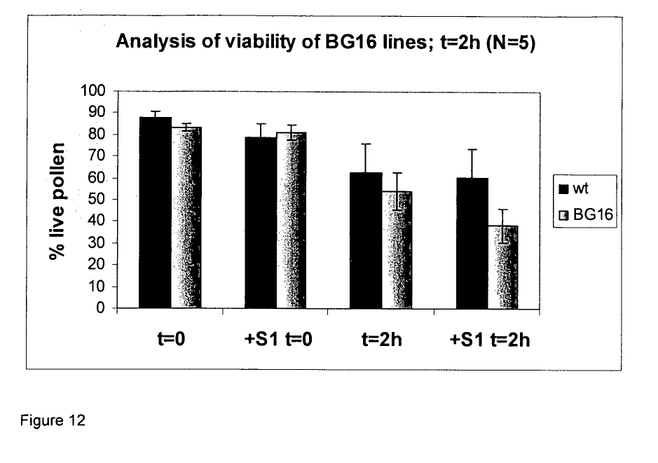

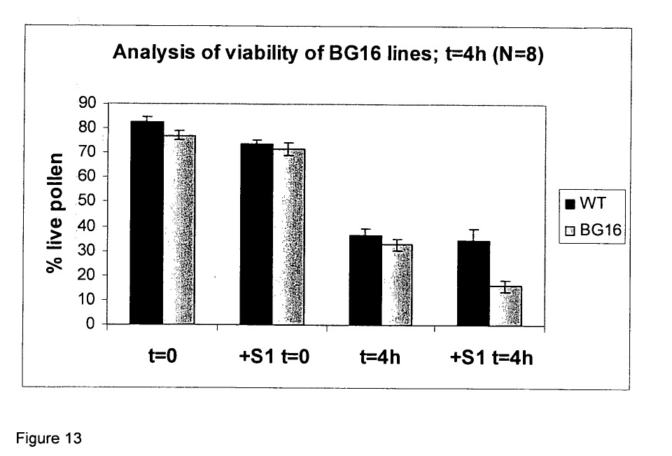

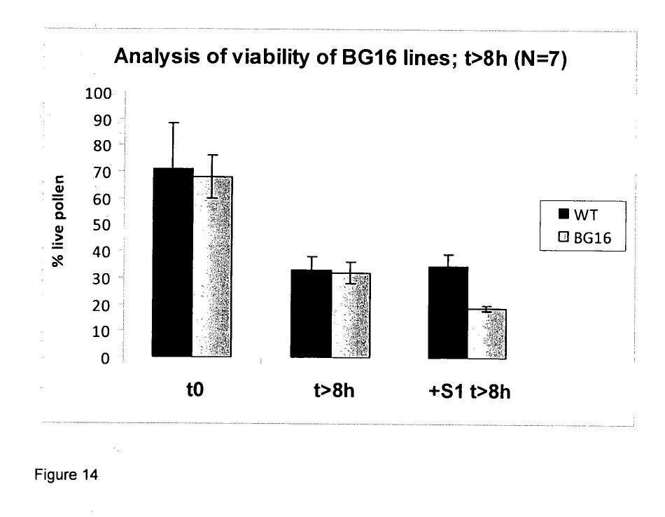

Pollen was grown in vitro and pre-treated with as-ODNs and s-ODNs for 1 hr (Moutinho et al. (2001) Sexual Plant Reproduction 14, 101-104; de Graaf et al. (2006) Nature 444, 490-493) prior to induction of SI with recombinant S₁, S₃ and S₈ proteins (Foote et al (1994) ibid). After 2 hours, pollen tubes were fixed in 2% paraformaldehyde and 50 pollen tube lengths were measured for each experiment. Each set of data presented in FIG. 7 is the combined result of three independent experiments, thus 150 pollen tubes in total (n=3).

Results

Pollen from plants of haplotype S₁S₃when challenged with incompatible S proteins was rescued from inhibition by PrpS₁ peptide (n=6) whereas randomized peptides based on the same amino acids had no effect (n=3). The demonstration that the PrpS₁ peptide blocked SI-induced pollen tube inhibition indicates a role for PrpS in regulating SI. As noted above, the antisense studies provided further evidence for this.

It was hypothesized that if a PrpS allele functions as the pollen S receptor, knockdown of its expression should result in alleviation of S-specific pollen tube inhibition. The pollen genotype/phenotype of plants carrying S₁S₃ alleles should theoretically be 50% S₁ and 50% S₃; those from plants carrying S₃S₈ should be 50% S₃ and 50% S₈. Thus, addition of as-ODN for PrpS₁ should only affect pollen carrying the S₁ allele, if the interaction is S-specific; furthermore, the theoretical maximum alleviation of inhibition by the as-ODN should be 50%, as only half of the pollen carries the S₁ allele.

SI induced strong inhibition of pollen tube growth (a 79% reduction in length compared to the controls) and a significant alleviation of this inhibition was observed in an incompatible combination in the presence of corresponding as-ODN and not with corresponding s-ODN (FIG. 7). Thus with pollen from plants carrying S₁S₃ alleles, SI induced strong inhibition of pollen tube length (22.1%, P=<0.001, n=300). Addition of as-PrpS₁-ODN gave a significant recovery of S protein-treated tubes, 58.3% increase in length (P=<0.001, n=150). As expected, the s-PrpS -ODN did not affect the SI response (P=0.591, n=150). S-specificity was also demonstrated; as-PrpS₈-ODN did not alleviate SI-induced inhibition when added to pollen from plants carrying S₁S₃ alleles (P=0.604, n=150). Using pollen from plants carrying S₃S₈ alleles, SI resulted in inhibited pollen tubes (19.8% of the control, n=300). Addition of the as-PrpS8-ODN alleviated the SI induced inhibition, giving a highly significant 100.3% increase in pollen tube length (P=<0.001, n=150), whereas there was little or no effect using as-PrpS₁-ODN or s-PrpS₈-ODN.

This data is consistent with the hypothesis that the PrpS alleles encode a transmembrane protein that mediates S-specific recognition and pollen inhibition and is thus the Papaver pollen S-determinant. Such proteins are very different from both the other S determinants so far identified: the Brassica pollen SCR/SP11 and the pollen F-box protein SLF/SFB of the S-RNase based SI system.

### Example 4

### Production of Transgenic Arabidopsis Thaliana with a P. Rhoeas SI System

The pistil S₁ and pollen S₁ genes of P. rhoeas obtained as above in a 42 kb genomic DNA fragment were used for the transformation study. 6.8 kb of the cloned S₁ locus was introduced into Arabidopsis thaliana plants, strain Columbia-0. This fragment contains the coding sequences for pistil S₁ and pollen PrpS₁ and 2.1 kb and 2.8 kb upstream promoter sequences respectively. Primers were designed with NotI restriction sites and the 6.8 kb S₁ fragment (S1g6.8) was obtained by PCR with the original 42 kb genomic DNA clone as a template. S1g6.8 was sequenced and cloned into the NotI site of pGreen0029, a binary Ti vector which confers kanamycin resistance in plants together with the transgene (Helens et al. (2002) Plant Mol. Biol. 42, 819-832).

Agrobacterium strain GV3101 carrying the T-DNA plasmid with S1g6.8 was used for stable transformation of Arabidopsis plants by the floral dip method (Clough & Bent. (1998) Plant J. 16, 735-743).

40 transformed Arabidopsis plants (T₁) were obtained by screening for kanamycin resistance of T₀ seed, and the presence of the transgene cassette S1g6.8 was confirmed by PCR of pistil S₁ and pollen PrpS₁ genes.

Since both transgenes present on the 6.8 kb T-DNA fragment are linked, in the absence of a recombination event between both genes, they can be regarded as a “single” T-DNA and should show Mendelian genetics. Single copy insertions present in the T1 generation of AtPrSI plants, without affecting pollen viability, would be expected segregate in a 3:1 ratio. Instead, when backcrossed to wild-type plants, pollen from these plants with single T-DNA insertion segregate 1:1.

Controlled pollinations by emasculation and manual pollinations, e.g. selfings and back-crosses to wild-type Arabidopsis plants, were carried out of several T1 Arabidopsis plants (BG06 Arabidopsis line: AtPrSI) and the segregation of kanamycin resistance analysed. In a normal situation, that is without changes in pollen and/or pistil functions, a 3:1 ratio (K+ vs. K−) is expected. However, a couple of the AtPrSI plants showed a 2:1 segregation ratio after self-pollination. Remarkably, pollination of these AtPrSI plants with wild type pollen or pollination of Arabidopsis Columbia-0 plants with AtPrSI pollen (heterozygous) showed segregation percentages as indicated above (1:1).

These results suggest that the transfer of the S1g6.8 cassette by AtPrSI pollen is affected in AtPrSI plants. The analysis, including silique size, of the T2 generation (10 plants per independent AtPrSI line) resulted in 70% ‘normal’ siliques (25-30 mm) and 30% with ‘smaller’ siliques (between 10 and 15 mm). Random segregation analysis of the T3 generations showed that all T2 generation plants with smaller siliques were homozygous for the T-DNA. These results indicate that pollen germination, tube growth and fertilization are affected in AtPrSI plants. The expression of Papaver rhoeas S₁ and PrpS₁ in Arabidopsis pistils and pollen, respectively, results in reduced seed set.

In a second set of experiments, a chimeric pollen PrpS₁-GFP gene was constructed for localization studies in pollen by transient expression and in pollen of stably transformed Arabidopsis plants. The chimeric gene comprises a green fluorescent protein (GFP) cDNA fused in frame with the 3′ end of the genomic copy of PrpS₁ (gPrpS₁). For the expression of this fusion protein use was made of the upstream promoter and 3′ terminator sequence of ntp303, a Nicotiana tabacum gene which as previously noted above is known to be specifically active in pollen, including Papaver rhoeas and Arabidopsis pollen. Again we made use of the pGreen0029 T-DNA plasmid for construction of the chimeric gene ntp303p-gPrpS1-GFP-3′utr303.

Agrobacterium mediated transformation of Arabidopsis Columbia-0 plants (see above) and screening for kanamycin resistance resulted in 35 T1 plants (BG16 Arabidopsis line) of which pollen was analyzed by fluorescence microscopy for the expression of PrpS₁-GFP fusion protein. Segregation analysis of a subset of these plants with single insertion of the transgene showed normal (3:1) ratios, demonstrating that the expression of PrpS₁-GFP did not affect Arabidopsis pollen germination, tube growth and fertilization.

Pollen from BG16 plants expressing PrpS,-GFP was used in an in vitro SI assay as developed for Papaver pollen (Foote et al. 1994 ibid) but adjusted for Arabidopsis pollen. Pollen was cultured on agarose in Arabidopsis pollen germination medium (GM). Pollen was grown in GM only or in the presence of recombinant Papaver rhoeas PrsS₁, PrsS₃, PrsS₈ or PrsS₃+PrsS₈ pistil proteins. Papaver pollen (S₁ S₈) grown in Arabidopsis pollen GM was used as a control.

The germination and pollen tube growth of BG16 pollen was unaffected in the presence of poppy recombinant PrsS3 or PrsS8 proteins or PrsS3+ PrsS8 proteins. Adding PrsS1 proteins resulted in a 50% reduction of pollen germination and all the non-germinating pollen grains were identified as PrpS₁-GFP expressing pollen. This experiment thus unequivocally demonstrated that the expression of PrpS₁ (PrpS₁-GFP) proteins in Arabidopsis pollen makes this pollen express a “PrpS1” phenotype that is recognized when it interacts with poppy PrsS₁ protein but not with PrsS3 or PrsS8 recombinant proteins. In other words, cognate sS1 and pS1 pairs render a “self-incomaptible” with the result that this pollen is inhibited.

### Example 5

### Materials Generated

(1) PrpS and PrsS Constructs put into Arabidopsis

This was done in accordance with the assistance of Dr. Barend de Graaf, Cardiff University.

Stable Transformants—Transgenic Arabidopsis Thaliana Lines:
T₀ is seed collected after floral dip, to screen for primary transformants.
T₁ is seed from primary transformant numbering: BG06.1, BG06.2 . . . etc
T₂ is seed from second generation plants from a single T1 plant: 6 1.1, 06.1.2 . . . etc sometimes with ‘HET’ (heterozygous) or ‘HOM’ (homozygous)

The following Arabidopsis thaliana (At) cell lines were generated (AtBG01, 02 etc.), see below, where Kan=Kanamycin; pGr0029=Pgreen (A vector has been developed where the three major components of a plasmid have been optimised for the improvement of plant transformation via Agrobacterium see http://www.pgreen.ac.uk/for details); STIG1=stigma-specific promoter from Celestina Mariani lab. (NL) [Stig1 is a 12-kD protein called stigma-specific protein 1 (STIG1)].

| | |
|---|---|
| Controls | |
| AtBG01 => pGr0029 control T (Kan in plants) | |
| AtBG04 => pGr0229 control T (Bar in plants) | |
| AtBG05 => pGr0029 control T (Kan) | |
| Whole S-locus (?including poppy promoters) | |
| AtBG06 => pGr-S1g6.4 (pGr0029/Kan) | whole S1 locus = |
| | pistil S1 & PrpS1 |
| AtBG08 => pGr-S1g6.4 (pGr0229/Bar) | whole S1 locus = |
| | pistil S1 & PrpS1 |
| Constitutive expression of PrpS (S35 promoter) | |
| AtBG02 => pGr35S-PrpS1A (in pGr0029/Kan) | PrpS1 |
| AtBG03 => pGr35S-PrpS1G (in pGr0029/Kan) | PrpS1 genomic |
| Tissue-specific expression of PrpS8 | |
| (PrpS1 promoter) | |
| AtBG14 => PrpS1p-PrpS8G-nosT | PrpS8 genomic with. S1- |
| (in pGr0029/Kan) | locus promoter |
| AtBG15 => PrpS1p-PrpS8G-nosT | PrpS8 genomic with. S1- |
| (in pGr0229/Bar) | locus promoter |
| GFP constructs so can track PrpS | |
| AtBG11 => PrpS1p-PrpS1G-GFP-303utr | PrpS1 genomic- |
| (in pGr0029/Kan) | GFP |
| AtBG16 => pGr-NGC-PrpS1G (in pGr0029/ | PrpS1 genomic- |
| Kan-NGC = bombardment cassette) | GFP w. |
| | ntp303promoter for |
| | high expression |
| AtBG17 => pGr35S-PrpS1G-YFP | PrpS1 genomic-YFP |
| (in pGr0029/Kan) | |
| Pistil (stigma) S8 (tissue-specific expression) | |
| AtBG12 => S1p-S8-nosT (in pGr0229/Bar) | stigma S8 |
| AtBG13 => STIG1p-S8-nosT (in pGr0229/Bar) | stigma S8 |
| with. STIG1 should be higher expression | |

### Example 6

### Construction of Four Further Expression Vectors

We have generated a number of additional lines of transgenic Arabidopsis for experimental studies. These comprise: pollen PrpS3-GFP and PrpS8-GFP, as well as pistil PrsS1 and PrsS3.

Having these Arabidopsis lines allowed us to perform reciprocal pollinations using combinations of different alleles of the gene to check specificity as well as function. The combinations include: PrpS1-GFP, PrpS3-GFP or PrpS8-GFP with PrsS1, PrsS3, PrsS8.

6.1 - Prps3-GFP

Construction of pGreen0029-NTP303-Prps3-GFP-303utr

The following flow diagram shows the protocol for construction of pGreen0029-NTP303-Prps3-GFP-303utr:

6.1.1 Ligation of cPrps3 into pDrive

Two primers of:

SEQ ID NO: 33:
5′ TCCCCATGGCACGAAATAGACATGC 3′
(sense, Nco I site underlined);
and
SEQ ID NO: 34
5′ TATGGATCCAGCCTCATTAGGACATGG 3′
(anti-sense, BamH I site underlined):

were used to get the PCR product of cPrps3. After ligation into pDrive, double enzymatic digest of Nco I/BamH I was performed to check the recombinant plasmid. Clone 2 was sequenced for confirmation. cPrps3 was ligated into pDrive and the recombinant plasmid was named as HZpDrive-Prps3.

6.1.2 Ligation of cPrps3 into pGreen0029 Expression Vector

The recombinant plasmid of pDrive-Prps3 was digested by Nco I/BamH I to release the cPrps3. cPrps3 was then ligated with the backbone of the expression vector pGreen0029-NTP303-Prps8-GFP-303utr digested with the same double enzymes. After ligation the recombinant plasmid was checked by digesting with Hpa I/Pst I.

FIG. 4 showed that the cPrps3 was probably ligated into pGreen0029. The following sequencing result also confirmed this (FIG. 5) and this recombinant plasmid was named as HZpNGC-Prps3.

6.2 Prps8-GFP

Construction of pGreen0029-NTP303-Prps8-GFP-303utr

The following flow diagram shows the protocol for construction of pGreen0029-NTP303-Prps8-GFP-303utr

6.2.1 Ligation of cPrps8 into pDrive

Two primers of:

SEQ ID NO: 35:
5′ TGCCCATGGCACGACATGCAATTGTTGTTC 3′
(sense, Nco I site underlined);
and
SEQ ID NO: 36:
5′ CGAAGATCTAACCTCAACACTACGGTGGG 3′
(anti-sense, Bgl II site underlined);

were used to get the PCR product of cPrps8. After ligation into pDrive, double enzymatic digest of Nco I/Bgl II was performed to check the recombinant plasmid. Clone 6 was sequenced for confirmation. cPrps8 was ligated into pDrive and the recombinant plasmid was named as HZpDrive-Prps8.

6.2.2 Ligation of cPrps8 into pGreen0029 Expression Vector

The recombinant plasmid of pDrive-Prps8 was digested by Nco I/Bgl II to release the cPrps8. cPrps8 was then ligated with the backbone of the expression vector pGreen0029-NTP303-Prps8-GFP-303utr digested with Nco I/BamH I. After ligation, the recombinant plasmid was checked by digesting with Nco I/BamH I (There is a BamH I site inside the cPrps8 sequence).

Electrophoresis result showed that the cPrps8 was ligated into pGreen0029. Sequencing confirmed this. This recombinant plasmid was named as HZpNGC-Prps8.

6.3 STIG1-S1

Construction of pGreen0229-STIG1-S1-nosT

The following flow diagram shows the protocol for construction of pGreen0229-STIG1-S1-nosT

6.3.1 Ligation of S1 into pDrive

Two primers of:

SEQ ID NO: 37:
5′ GCCCCATGGGCAACATATTTTATGTTATTGTGCTG 3′
(sense, Nco I site underlined);
and
SEQ ID NO: 38:
5′ AATGCGGCCGCTCAGGTTCGACCTTCCTTCCTTTC 3′
(anti-sense, Not I site underlined);

were used to get the PCR product of S1. After ligation into pDrive, double enzymatic digest of Nco I/ Not I was performed to check the recombinant plasmid. And then the third clone was chosen for sequencing for further conformation. S1 was ligated into pDrive and the recombinant plasmid was named as HZpDrive-S1.

6.3.2 Ligation of S1 into pGreen0229 Expression Vector

The recombinant plasmid of pDrive-S1 was digested by Nco I/Not I to release the S1. S1 was then ligated with the backbone of the expression vector pGreen0229-STIG1-S8-nosT digested with the same double enzymes. After ligation the recombinant plasmid was checked by digesting with Nco I/Not I. Electrophoresis results showed that the S1 was ligated into pGreen0029 (FIG. 13). Sequencing confirmed this. This recombinant plasmid was named HZStig1p-S1-nos.

6.4 STIG1-S3

Construction of pGreen0229-STIG1-S3-nosT

The following flow diagram shows the protocol for construction of pGreen0229-STIG1-S3-nosT

6.4.1 Ligation of S1 into pDrive

Two primers of:

SEQ ID NO: 39:
5′ GCCCCATGGGCAAGATATTGTGCGTTATTGTGCTTC 3′
(sense, Nco I site);
and
SEQ ID NO: 40:
5′ AATGCGGCCGCTCAGACTTCCTTCTCACCCATTC 3′
(anti-sense, Not I site);

were used to get the PCR product of S3. After ligation into pDrive, double enzymatic digest of Nco I/ Not I was performed to check the recombinant plasmid. And then the third clone was chosen for sequcencing for further conformation. S3 was ligated into pDrive and the recombinant plasmid was named as HZpDrive-S3.

6.4.2 Ligation of S3 into pGreen0029 Expression Vector

The recombinant plasmid of pDrive-S3 was digested by Nco I/Not I to release the S3. S3 was then ligated with the backbone of the expression vector pGreen0229-STIG1-S8-nosT digested with the same double enzymes. After ligation the recombinant plasmid was checked by digesting with Nco I/Not I. Electrophoresis results showed that the S3 was ligated into pGreen0029. Sequencing confirmed this and this recombinant plasmid was named as HZStig1p-S3-nos.

### Example 7

### Transformation of the Four Constructs into Agrobacterium

These 4 constructs together with pSop plasmid were transformed into competent Agrobacterium cells. 3-4 days later, monoclones were found. Several monoclones of each construct were selected for culture overnight in LB+Kan⁺+Tet⁺+Rif⁺ liquid medium and then extracted plasmids from these transformants. Then the plasmids from Agrobacterium were transformed into DH5α. Monoclones of DH5α transformants were selected for culture in LB+Kan⁺+Tet⁺Rif⁺ liquid medium and extracted plasmids from these transformants again. Finally, the plasmids from DH5α transformants were checked by digesting with enzymes. The 4 constructs were all successfully transformed into Agrobacterium.

These constructs were called HZDH5αXXX (PrpS3, 8, S1, S3).

### Example 8

### Infection of Arabidopsis via Floral Dipping

After floral dipping, we collected seeds, and selected the transformants via antibiotic and basta resistances.

### Example 9

### Seed and Progeny

We now have T0 and T1 seed of all of these HZ-lines.
We are currently selecting the T1 transformants for antibiotic and basta resistances.
These have been tested for inserts using PCR, and we will shortly test for level of expression in flowers using RT-PCR.

### Example 10

### Analysis of Transformed Lines of Examples 6-9

To date, all analysis has been carried out on At line BG16 (carrying PrpS1-GFP).

Methods

Measurement of Length and % Germination

Pollen grains from the second transformant lines of AtBG16 (BG16.7.4, BG16.8.3, BG16.15.1, BG16.15.2 and BG16.15.3), generated by Barend de Graaf, and wild type Col0 were germinated and pictures taken under the 10× objective at time intervals using a Nikon eclipse Tε300 inverted microscope and the Nikon Imaging Software elements BR3.0 program. The experiment included six technical replicates and two biological replicates. Randomised samples of one hundred pollen tubes/grains were analysed per biological replicate (n=100×2) and the number of grains and tubes were counted and the length of the tubes was measured using the NIS elements BR3.0 software package.

In Vitro SI Induction

The same type of set-up as used for Papaver “In vitro SI induction” was used; see (Snowman, 2002). This comprises germinating pollen in vitro on slides in the presence of recombinant PrsS proteins. In an incompatible combination this gives an incompatible response, i.e. pollen rejection and inhibition. Several downstream events are triggered in incompatible pollen and are used as markers for SI in Papaver (see (Geitmann et al., 2000; Thomas and Franklin-Tong, 2004; Bosch and Franklin-Tong, 2007; Poulter et al., 2008), and (Bosch et al., 2008) for a recent review.

For the experiments described below, the in vitro SI induction was as follows: pollen from BG16.8.3, wild type Col0 and P. rhoeas plants were germinated in the appropriate pollen growth medium on multiwell slides. Recombinant PrsS (see below) was added where required to a final concentration of 20 μg ml-¹. PrsS₁ proteins were denatured by boiling for 5 minutes. The test experiment consisted of BG16.8.3 grown in the presence of S₁ proteins. The controls included Col0 alone, Col0 with PrsS, and PrsS₈ proteins, BG16.8.3 alone, BG16.8.3 with denatured PrsS₁ and BG16.8.3 with PrsS₈ protein. P. rhoeas were grown alone, in the presence of S₁ and in the presence of both PrsS₁ and PrsS₈. The experiment had two technical replicates and three biological replicates (n=100×3).

PrsS Proteins

Recombinant PrsS proteins (PrsS₁ and PrsS₈) (Foote et al., 1994; Walker et al., 1996; Kakeda et al., 1998) were dialysed overnight at 4° C. from Tris buffer into 13.5% growth medium. To determine which concentration of PrsS was needed to induce SI, the PrsS-proteins were diluted to a final concentration of 5 μg ml⁻¹, 10 μg ml⁻¹ and 20 μg ml⁻¹ when added to the germinating BG16.8.3 pollen in vitro.

Microscopy and Scoring Criteria

Randomised samples of 100 pollen grains/tubes were analysed per biological replicate under the 10× objective using a Nikon ε400 microscope and the Cell^{P} Soft Imaging System for Life Science microscopy program. Pictures were taken simultaneously under brightfield (with an exposure time of 50 ms at a CCD gain of 0) and with 492/18× Single Band Blue exciter for FITC (with an exposure time of 1 s at a CCD gain of 200). The number of pollen grains and tubes were scored according to the scoring criteria, as shown in Table 1 below.

Evans Blue Staining

Viable cells are not stained. Non-viable (dead) cells are stained dark blue with this viability stain. Pollen was stained with 0.05% Evans blue for 15 minutes. After incubation, samples were washed to remove excess dye. 20 μL of sample was mounted on a microscope slide and pollen was visualised using brightfield microscopy (Nikon Eclipse Tε300). Scoring categories were: unstained (live), heavily stained (dead). Each experiment was repeated 3× with 180 pollen counted each time.

Statistical Analysis

X² test was used for segregation analysis and one-way analysis of variance (ANOVA) was used to test for the significant difference between experiment and controls.

Results

Germination and Length

Purpose: Before any studies could be undertaken, it was important to establish whether the transgenic pollen behaved as normally as wild type Col0 pollen.

The germination of A. thaliana pollen is a laborious process and showed considerable variation. It is well known that A. thaliana pollen is notoriously difficult to grow, with highly variable results (Johnson-Brousseau and McCormick, 2004). Pollen was germinated on liquid pollen growth medium and the percentage germination and lengths of pollen tubes were recorded, using the scoring criteria indicated in Table 1. We compared if the AtBG16 lines differed significantly from the wild type ColO. One-way ANOVA confirmed that the percentage germination and mean length of pollen tubes were not significantly different from the wild type Col0 (p=0.477 and p=0.996 respectively, n=100×2). Hence, the inserted genes did not affect the behaviour of the pollen grains, that is, pollen hydration and germination were as normal wild type.

Segregation Analysis

The primary transformants of the AtBG16 lines were believed to be heterozygous and a segregation analysis was performed to verify this. Since the transgenic plants had kanamycin resistance marker to distinguish them from untransformed plants, the seeds from the primary transformants were sterilised and plated on growth medium containing the kanamycin antibiotic. Those that were homozygous or heterozygous for the inserted gene would grow on the kanamycin growth medium, while those that lacked the transgene would die.

X² test confirmed that the primary transformants had a normal expected segregation ratio of 3:1 (X²=5.52, p>0.05, n=100×3). Pollen containing the PrPS₁ transgene behaved like wild type Col0 pollen and had no problem in hydrating, germinating and fertilising ovules to produce viable seeds. Therefore, the inserted PrpS₁ gene linked to GFP did not interfere with the normal functioning of pollen grains.

In Vitro SI Assay

In order to assess the functional role of the PrPS₁ gene from P. rhoeas in A. thaliana, pollen from BG16.8.3 was challenged with recombinant PrsS₁ proteins (20 μg ml⁻¹) in the appropriate pollen growth medium, using the “in vitro SI induction”, as described earlier. Denatured S₁ proteins and S₈ proteins were included to verify that active S proteins were responsible for SI induction and that SI response was triggered in an S-specific manner.

As a control, to verify that the PrsS proteins were functional in the in vitro SI system, germination of poppy pollen was tested. This was significantly reduced with high concentration of S₁ and S₈ proteins (p=0.000, n=100×3). This is illustrated in FIG. 8. Since pollen from P. rhoeas, carrying the S₁- and S₈-haplotype, was used, it was expected that in the absence of S proteins, the majority of tubes would germinate (50%±0) and that addition of S₁ protein would inhibit the PrPS₁ expressing pollen tubes alone, such that only the PrpS₈ expressing pollen tubes could germinate (29.7%±0.577, p=0.000, n=100×3). The best interpretation for this is that those pollen which expressed PrPS₁, bound their cognate PrsS₁ proteins and were inhibited, while the tubes that expressed PrPS₈ were not inhibited because PrsS₁ protein was not their cognate ligand. They therefore grew normally. Addition of both PrsS₁ and PrsS₈ proteins caused most of the tubes to be inhibited (1%±1, p=0.000, n=100×3), as predicted, as SI was induced.

Col0 was unaffected by PrsS proteins (FIG. 9, p=0.760, n=100×3). Since it was untransformed, it did not express the PrPS gene and was therefore self-compatible and did not express GFP. Moreover, the percentage germination was not significantly different between BG16.8.3 and Col0 (p=0.400, n=100×3).

FIG. 10 summarises the effect of recombinant PrsS proteins on the germination of BG16.8.3 pollen (analysis of all pollen). It was expected that in the presence of PrsS₁ protein, the percentage germination of BG16.8.3 pollen would decrease, but not all of the pollen would be affected, as these plants are segregating (as they are not homozygous lines), and only about 50% of the BG16.8.3 pollen tubes were expected to express the PrPS₁ gene fused to GFP. Thus, only the PrPS₁ expressing pollen tubes would be expected to be inhibited. Although the percentage germination of BG16.8.3 did decrease upon addition of PrsS₁ (see FIG. 10) it was not a statistically significant fall in germination (p=0.315, n=100×3). This is probably because only half of all pollen is expressing PrPS₁ and only these would be expected to be inhibited.

Denatured S₁ protein was expected to be inactive and incapable of inducing SI; there was no significant difference in germination of BG16.8.3 pollen (p=0.720, n=100×3). Similarly, the percentage germination of BG16.8.3 was expected to be unaffected by PrsS₈ protein because BG16.8.3 was transformed with PrPS₁ gene, which was inhibited specifically with PrsS₁ proteins and not PrsS₈ proteins; there was no significant difference observed in the percentage of germination of BG16.8.3 pollen tubes (p=0.804, n=100×3).

In summary, FIG. 10 shows the % germination of pollen tubes with different S proteins. There was no significant difference observed in the percentage germination of BG16.8.3 pollen tubes subjected to various treatments (p=0.315, n=100×3).

As predicted (see FIG. 11), when we focused our analysis on just the percentage of germinated tubes which expressed the PrPS₁-GFP, the effect was much better than that in FIG. 10. We plotted only the pollen that was seen to be expressing GFP (assessed by microscopy). When we did this, no transgenic pollen tubes expressing PrPS₁ germinated in the presence of active S₁ proteins. This was a highly significant result (p=0.000, n=100×3). In the presence of PrsS₁ proteins, no transgenic tubes that expressed GFP germinated because they expressed the PrPS₁ gene (FIG. 10). This could be interpreted as being due to the PrPS₁ interacting with the PrsS₁ proteins in the growth medium, resulting in the arrest of pollen tube growth.

Those tubes that did grow in the presence of PrsS₁ proteins lacked the PrPS₁ gene and did not express GFP. Thus, in the absence of S₁ proteins, or in the presence of PrsS8 protein or biologically inactive PrsS1, inhibition of PrPS₁-GFP pollen was not observed, and there was no significant difference in the percentage of germination of BG16 pollen tubes expressing PrPS₁-GFP in the other control treatments (p>0.05, n=100×3). Denatured PrsS₁ protein was biologically inactivate and incapable of inducing SI and also acted as a control. As expected, the effect of denatured PrsS₁ proteins on BG16.8.3 tubes was not significantly different from untreated samples in the (p=0.963, n=100×3). Similarly, the percentage of germinated BG16 pollen tubes expressing GFP was expected to be unaffected by PrsS₈ proteins because BG16.8.3 was transformed with PrPS₁ gene and PrsS₈ proteins had no significant effect on the percentage of GFP-expressing BG16.8.3 vs untreated samples (p=0.124, n=100×3). This clearly demonstrates the S-specificity of the effect of the pistil S determinant PrsS1 on BG-16 GFP-expressing pollen PrpS₁ with its complementary PrsS₁ ligand.

In summary, transgenic A. thaliana lines, expressing the PrPS₁ gene fused to GFP (a BG16 line), show that SI is mediated by PrPS expression in A. thaliana pollen in the presence of active recombinant poppy PrsS₁ proteins. Recombinant PrsS proteins inhibit pollen tube growth in an S-specific manner. The poppy SI system which was introduced into A. thaliana worked in the presence of its active complementary PrsS protein alone. This confirms that the poppy PrPS gene is functional in A. thaliana. The recombinant poppy pistil PrsS₁ protein and the pollen protein, PrpS₁ expressed in A. thaliana alone were sufficient to induce S-specific pollen inhibition in A. thaliana. This suggests that the events downstream of the interaction are well conserved in unrelated species, and are likely be present in all species where the SI system might be transferred to. Thus, this data provides a strong basis for believing that the poppy SI system can be transferred to other species.

We have also performed other studies on line BG16 (Pollen PrpS1) to further establish that poppy SI is functional in Arabidopsis, using recombinant PrsS proteins to challenge pollen grown in vitro. These studies are also using the “in vitro SI” system (addition of recombinant PrsS1 protein to induce SI in PrpS1-containing pollen).

### Example 11

### Viability Results—Effect of Addition of Recombinant PrsS1 Protein on AtBG16 Pollen

We used Evans blue to test for viability of At-BG16 (containing PrpS1) pollen grains that were given an “in vitro SI” treatment (challenged with recombinant PrsS in vitro-see above for methods). This resulted in a reduction in viability of BG16 with PrsS1 added compared to wild type pollen challenged.

FIG. 12 shows the viability of Arabidopsis wild type pollen (blue bars) and pollen expressing PrpS-GFP (yellow bars) at t=0 h and t=2 h in the absence and presence of poppy recombinant female S-determinant PrsS₁.

FIG. 13 shows the viability of Arabidopsis wild type pollen (black bars) and pollen expressing PrpS-GFP (yellow bars) at t=0 h and t=4 h in the absence and presence of poppy recombinant female determinant PrsS₁.

FIG. 14 shows the viability of Arabidopsis wild type pollen (dark green bars) and pollen expressing PrpS-GFP (yellow bars) at t=0 h and t>8 h in the absence and presence of poppy recombinant female determinant PrsS₁.

In Summary:
BG16+PrsS₁ has 35 times lower viability compared to WT+S₁ pollen at t=2 h.
BG16+PrsS₁ has 53 times lower viability compared to WT+S₁ pollen at t=4 h.
BG16+PrsS₁ has 45 times lower viability compared to WT+S₁ pollen at t>8 h.

Decreased viability in Arabidopsis pollen expressing PrpS₁ suggests that death is triggered in some of the BG16 pollen (again, note that only 50% of the pollen is expected to carry PrpS1, so only half will be expected to respond).

This suggests that programmed cell death (PCD) may well be triggered by “in vitro SI” in BG16, further suggesting that poppy SI mechanisms may be triggered in Arabidopsis when the right PrpS-PrsS combination is used.

This will be explored next, together with tests for specificity (challenge with recombinant PrsS3 or 8).

### Example 12

### In Vivo Pollination Data—Aniline Blue Assessment

Analysis of pollinations using aniline blue staining to visualize (UV×10 and Bright Field×10) pollen grains and tubes, allowed us to assess pollen rejection for the different crosses.

Pistils of Arabidopsis line HZ1 were emasculated prior to anthesis and left to mature. They were then pollinated with BG16 pollen and left overnight. Pollinations were made in pair-wise combinations, so we used exactly the same BG16 pollen (prpS1) on both wt and HZ stigmas to give a proper comparison. Controls comprised wildtype pistils pollinated with BG16 pollen to check that pollen was viable and functioning properly. HZ pistils were also pollinated with wildtype pollen for controls to ensure they were normal and functional.

The pistils were then harvested and placed in aniline blue, left for several hours and then viewed using UV microscopy.

Crosses were attempted with HZ1 independent transfomants (Hz1.3 and Hz1.5): HZ1.3×BG16 and HZ1.5×BG16. Each cross was replicated with BG16 pollen on two HZ stigmas; the control was BG16 pollen on a wild type stigma. The results for the wt stigma cross with BG16 pollen was normal, showing lots of long (stained) pollen tubes growing down into the style. However, the results for the HZ1.3 stigmas showed markedly different staining patterns to the control pollination (BG16×wt), even though the pollen is all from the same sample. In the HZ1.3 stigmas, the staining was concentrated at the top of the pistil, showing evidence of inhibited pollen grains and pollen tubes providing evidence that at least some of the pollen was inhibited. As BG16 plants are heterozygous, only 50% of pollen produced will be PrpS-GFP, so only 50% should be inhibited. Thus, we expect a “half-compatible response, with 50% pollen inhibited and 50% growing normally. There is certainly evidence of inhibition in this HZ1.3×BG16 cross, especially if one compares it with wild type controls.

Similar patterns were seen for the HZ1.5×BG16 cross and its accompanying control (wt stigma×BG16 pollen).

The data shows that there is rejection of cognate PrpS1 pollen when a PrsS1 pistil (the HZ1 line expressed PrsS1) is pollinated with BG16 (again expressing PrpS1) pollen. In some instances the rejection appears to be very marked. This data shows that the BG 16 pollen and, therefore, the present SI system is functional in vivo.

### Example 13

### In Vivo Pollination Data Using Combinations of PrpS1-GFP, PrpS3-GFP or PrpS8-GFP with PrsS1, PrsS3, PrsS8

Example 12 is repeated in vivo using combinations of PrpS1-GFP, PrpS3-GFP or PrpS8-GFP with PrsS1, PrsS3, PrsS8 in pairwise comparisons respectively.

Crosses in different PrpS-PrsS combinations were made to obtain expected “self”, “cross” and control (vs wt) combinations. For example (using plants with PrsS as female recipient, and plants with PrpS as male pollinator): PrsS1×PrpS1, PrsS3×PrpS3 and PrsS8×PrpS8 are expected to be incompatible (no seed); PrsS1×PrpS3, PrsS1×PrpS8, PrsS3×PrpS8 are expected to be compatible (seed), and these lines vs wild type should all set seed.

Crosses can also be performed between the lines produced, to generate lines with the S-locus allelic pairs together. We can then perform further functional crossing tests, as outlined above, on these lines.

### Example 14

### Biochemical/Cell Biology Analysis

The purpose of this experiment is to ascertain if key features of SI are triggered. This approach can be used to identify whether key features such as programmed cell death (PCD) of incompatible pollen of Poppy SI are triggered in incompatible combinations of PrpS-PrsS in Arabidopsis. This provides evidence that the mechanisms triggered by SI are triggered in the “self” (incompatible) combination interaction, i.e. confirming that rejection is due to this effect.

This is tested using combinations of cell lines containing PrpS1-GFP, PrpS3-GFP or PrpS8-GFP with PrsS1, PrsS3, PrsS8, respectively, in pairwise comparisons. Where “cognate” pairwise combinations are used, i.e. “incompatible” combinations (eg sS1 & pS1, sS3 & pS3, sS8 & pS8) these result in PCD, and where controls were employed using non-cognate pairs, i.e. compatible combinations (e.g. sS1 & pS3, sS1 & pS8, sS1 & pS8, sS3 & pS1, sS3 & pS8, Ss8 & pS3) or wild-type controls, no PCD results.

The following approaches were used:

(1) Test for cell viability. This is a quick test to check if cells were dead, as a precursor to testing for PCD. This employs viability stains, such as Evans blue or propidium iodide (dead cell stains), or fluorescein diacetate (a live cell stain). Incompatible combinations result in cell death, while compatible or controls do not undergo PCD, so no dead cells above basal background levels are detected.

(2) TUNEL assays &/or caspase assays are used to assess if programmed cell death (PCD) is triggered in an incompatible combination (and not a compatible combination). TUNEL protocols use procedures to assess levels of DNA fragmentation, which is an end-product of PCD, as described in Thomas et al (2004). Caspase assays involve production of cell extracts after the interaction and measurement of these extracts to ascertain whether caspase activity is triggered. This is, detected by using a fluorescent substrate to assess the cleavage activity (using methods as described in Bosch et al (2007). Incompatible combinations result in PCD so stain positive with TUNEL, while compatible or controls do not undergo PCD, so are TUNEL-negative.

(3) Alterations in actin cytoskeleton are triggered specifically in incompatible and not in compatible combinations. Typical experiments use rhodamine-phalloidin staining of fixed pollen tubes and are visualized using fluorescence microscopy. Incompatible reactions are characterized by actin depolymerisation and formation of large actin foci later (see Geitmann et al 2000; Snowman et al 2002). Incompatible combinations result in deploymerization and later formation of large actin foci, and compatible or controls display normal actin arrays.

### Example 15

### Other Cell Lines

Further cell lines (we have already generated one called BG03), with constitutive expression of PrpS1, have been used to test for signs of PCD after addition of poppy recombinant PrsS1 protein in leaf tissue. This ascertains if this system can be used in other cells types to trigger PCD or death. This experiment confirms whether the system might be used for ablation of particular cell types if the correct tissue-specific promoter is used. Preliminary data (not shown) indicates that there is an SI effect.

### Example 16

### Other Plant Species

Example 12 was performed in Arabidopsis. It is repeatable in other plant species, for instance barley. Suitable barley cells lines expressing the pistil and pollen S alleles are created, using the constructs based on HZ and BG: WH1=PrsS3, WH2=PrpS3, WH3=PrpS1 line. These were transferred into barley using Agrobacterium-mediated transformation, as described above.

The pistil and pollen S allele lines are then crossed as described in Example 12 and analysis of pollinations performed using aniline blue staining and fluorescence microscopy to visualize pollen grains and tubes. This, allowed an assessment of pollen rejection for the different crosses. Pistils of barley WH1 (carrying PrsS3) were emasculated prior to anthesis and left to mature. They were then pollinated with the barley transgenic line WH2 (carrying PrpS3) or WH3 (carrying PrpS1) and bagged to prevent contamination with any other pollen source. Controls comprised wildtype pistils pollinated with WH2 or WH3 to check that pollen was viable and functioning properly. WH1 pistils were also pollinated with wildtype pollen for controls to ensure they were normal and functional. Pollinated pistils were left for 24 hours. Some pistils were then harvested and placed in aniline blue, left for several hours or overnight and then viewed using UV microscopy to assess pollen rejection. Other pistils were left to set seed.

It is predicted that the crosses should have the following outcomes:

Assuming WH lines are heterozygous for PrsS and PrpS, rejection should be partial (50%). WH1×WH2 have 50% rejection of pollen, and lower seed set. WH1×WH3 have no pollen inhibition and 100% seed set. All pollinations with wt pollen or pistils have no pollen rejection and 100% seed set. This would show that the present SI system is functional in vivo in barley, an important example of a crop plant.

### REFERENCES

All references cited herein are incorporated by reference.
Bosch M, Franklin-Tong V E (2007) Temporal and spatial activation of caspase-like enzymes induced by self-incompatibility in Papaver pollen. Proceedings of the National Academy of Sciences USA 104: 18327-18332
Bosch M, Poulter N S, Vatovec S, Franklin-Tong V E (2008) Initiation of Programmed Cell Death in Self-Incompatibility: Role for Cytoskeleton Modifications and Several Caspase-Like Activities. Molecular Plant 1: 879-887
Foote H C C , Ride J P, Franklintong V E, Walker E A, Lawrence M J, Franklin F C H (1994) Cloning and Expression of a Distinctive Class of Self-Incompatibility (S) Gene from Papaver rhoeas L. Proceedings of the National Academy of Sciences of the United States of America 91: 2265-2269
Geitmann A, Snowman B N, Emons A M C, Franklin-Tong V E (2000) Alterations in the actin cytoskeleton of pollen tubes are induced by the self-incompatibility reaction in Papaver rhoeas. Plant Cell 12: 1239-1251
Kakeda K, Jordan N D, Conner A, Ride J P, Franklin-Tong V E, Franklin F C H (1998) Identification of residues in a hydrophilic loop of the Papaver rhoeas S protein that play a crucial role in recognition of incompatible pollen. Plant Cell 10: 1723-1731
Poulter N S, Vatovec S, Franklin-Tong V E (2008) Microtubules Are a Target for Self-Incompatibility Signaling in Papaver Pollen. Plant Physiol. 146: 1358-1367
Snowman B N, Kovar, D. R., Shevchenko, G., Franklin-Tong, V. E., and Staiger, C. J. (2002) Signal-mediated depolymerization of actin in pollen during the self-incompatibility response. Plant Cell 14: 2613-2626
Thomas S G, Franklin-Tong V E (2004) Self-incompatibility triggers programmed cell death in Papaver pollen. Nature 429: 305-309
Walker E A, Ride J P, Kurup S, FranklinTong V E, Lawrence M J, Franklin F C H (1996) Molecular analysis of two functional homologues of the S-3 allele of the Papaver rhoeas self-incompatibility gene isolated from different populations. Plant Molecular Biology 30: 983-994

1. A method of obtaining plants which exhibit self-incompatibilty (SI), or in which SI is inducible, which comprises transforming plants or cultured plant cells with both (i) a pollen S allele of a Papaver S locus or a functional variant thereof and (ii) a pistil S allele of said Papaver S locus or a functional variant thereof, said pollen and pistil S alleles encoding respectively pollen and pistil proteins of a Papaver SI system which prevents self-pollination, and, if need be, further generating plants from transformed cultured cells.

2. A method as claimed in claim 1, wherein said alleles are present in a complete native Papaver S locus.

3. A method as claimed in claimed in claim 1, wherein a PrpS and pistil S allele pair of Papaver rhoeas are employed.

4. A method as claimed in claim 1, wherein the PrpS₁ and pistil S₁ allele pair of P. rhoeas are employed.

5. A method as claimed in claim 1, wherein said plants or cells to be transformed contain, or are simultaneously transformed with, a transgene for a further desired characteristic.

6. A method as claimed in claim 1, which further comprises controlled pollination of transformed plants to obtain plants stably transformed and homozygous for said pollen S allele or a functional variant thereof and said pistil S allele or a functional variant thereof.

7. A method as claimed in claim 6, which further comprises crossing said plants homozygous for said pollen S allele or a functional variant thereof and said pistil S allele or a functional variant thereof to obtain F1 hybrid seed and optionally growing from said seed F1 hybrid plants.

8. A method according to claim 1, wherein the plant is an ornamental flower or a crop plant selected from the group consisting of: soybean, cotton, canola, corn, wheat, sunflower, sorghum, alfalfa, barley, millet, rice, fruit and vegetable crops.

9. A plant obtained by a method as claimed claim 1, which exhibits SI, or in which SI can be induced, by virtue of said pollen S allele or functional variant thereof and said pistil S allele or functional variant thereof.

10. A plant as claimed in claim 9, which is homozygous for said alleles conferring SI.

11. A plant as claimed in claim 10, which is an ornamental plant, seed therefor and cut flowers derived therefrom.

12. Seed for a plant as claimed in claim 11.

13. F1 hybrid seed obtained in accordance with claim 7, and F1 hybrid plants derived therefrom.

14. Cultured plant cells transformed in accordance with claim 1.

15. A kit of polynucleotides, for use in a method as claimed in claim 1, comprising two polynucleotides:
one polynucleotide having a pollen S allele of a Papaver S locus or a functional variant thereof; and
the other polynucleotide having a pistil S allele of said Papaver S locus or a functional variant thereof,
said pollen and pistil S alleles encoding respectively pollen and pistil proteins of a Papaver SI system which prevents self-pollination.

16. A kit as claimed in claim 15, comprising;
a vector, such as Agrobacterium, said vector comprising said both polynucleotides; or
two vectors, such as Agrobacterium, each vector comprising one or other of said polynucleotides.

17. A kit as claimed in claim 16, comprising instructions for use.

18. A method for targeted plant cell ablation in a plant tissue wherein the cells of the tissue express, as a heterologous protein, the pollen S protein component of a Papaver SI system or a functional analogue thereof, which comprises contacting said cells with, or co-expressing in said cells, a Papaver pistil S protein or functional analogue thereof whereby programmed cell death occurs.

19. A method as claimed in claim 18, wherein said pollen S protein component is the pollen S protein component of an SI system of P. rhoeas.

20. A method as claimed in claim 18, wherein said plant tissue is an ectopic tissue of a plant.

21. A method as claimed in claim 18, wherein pollen which expresses as a heterologous protein the pollen S component of a Papaver SI system is targeted to achieve inhibition of pollen growth.
